(19) United States
(12) Patent Application Publication
(10) Pub. No.: US 2025/0222454 A1
(43) Pub. Date: Jul. 10, 2025

(54) Microfluidic Device with On-Demand Droplet Trapping and Release for Single Cell Analysis

(71) Applicant: Northeastern University, Boston, MA (US)

(72) Inventors: Tania KONRY, Boston, MA (US); Giovanni Stefano UGOLINI, Allston, MA (US)

(21) Appl. No.: 18/850,819

(22) PCT Filed: May 5, 2023
(86) PCT No.: PCT/US2023/021237

§ 371 Date: Sep. 25, 2024

Related U.S. Application Data
(60) Provisional application No. 63/338,508, filed on May 5, 2022.

(51) Int. Cl. B01L 3/00; C12M 1/34

(52) U.S. Cl. CPC B01L 3/502784; B01L 3/502738; B01L 3/502761; C12M 41/46; B01L 2200/027; B01L 2200/16; B01L 2300/0867; B01L 2300/0887; B01L 2300/10; B01L 2300/123; B01L 2400/084

(57) ABSTRACT

Microfluidic devices and methods are provided for single-cell analysis, isolation, and preparation for immunotherapy. The technology allows functional phenotyping by observing cell-cell interactions and then cells with desirable features or probing the underlying biology. A droplet-based microfluidic device is capable of trapping droplets and releasing them selectively using microvalves. Each droplet can encapsulate effector cells, such as natural killer cells (NK cells) and target cells, such as tumor cells, for real-time monitoring of burst kinetics and spatial coordination during killing of tumor cells by individual NK cells. The technology also allows for interrogating interactions and real-time monitoring of kinetics and then recovering live cells on demand for single-cell genomic or proteomic analysis.

### CROSS REFERENCE TO RELATED APPLICATIONS

This application claims priority to U.S. Provisional Appl. No. 63/338,508, filed on 5 May 2022, which is hereby incorporated by reference in its entirety.

### BACKGROUND

Adoptive cell transfer-based immunotherapies rely on administering highly cytotoxic immune cells, particularly T cells and natural killer (NK) cells^{1,2}. Immune cells are highly heterogeneous and highly dynamic during adaptive response. It has been suggested that patients' inherent sensitivity or response to therapy is influenced by molecular heterogeneity and polymorphic characteristics of immune effector cells²⁻⁴. Ensuring isolation and expansion of immune cells with high anti-tumour activity presents a considerable challenge^{3,5}. NK and T cells often interact with targets transiently, forming short-lived dynamic immunological synapses^{6,7}, which cannot be detected by end-point assays but require a dynamic, time-resolved, analytical method. It has been hypothesized that the duration of synaptic contact between immune and cancer cells is a key factor in regulating downstream effector function. In other words, the efficiency of cell regulation and latter target cytotoxicity could be modified by mechanisms that enhance or decrease the intercellular synaptic contact periods. This necessitates functional phenotyping in a non-destructive manner to recover immune cells for downstream analysis. Current experimental paradigms either track individual cells functionally or measure genetic/protein levels of cell populations, which may not provide accurate results due to the lack of direct correlation between genetic and physiological states⁸. This severely limits understanding of interactive and secretion dynamics from single cells and small cohorts of heterogeneous cells at single-cell resolution. The state of the art provides no single-cell systems that address all of the above-described biology. However, there are various in vitro tools for single immune cell analysis available; these existing technologies have inherent limitations that affect cell retention, mobility, interactions and long-term real-time monitoring of kinetics and cell recovery on demand⁹⁻¹⁹.

Droplet microfluidics has shown the ability to encapsulate single cells²⁰⁻²⁴, and is well suited for studying single-cell interactions. Droplet microfluidics uses device geometries such as T-junctions²⁵, flow-focusing²⁶ and co-flow²⁷ to generate droplets containing cells. However, it is essential to manipulate these droplets using splitting²⁸⁻³⁰, sorting³¹⁻³³, trapping³⁴⁻³⁶ and merging^{37,38}. Selectivity in generation³⁹, splitting⁴⁰, merging⁴¹ and sorting⁴² is available in the literature, but selectively releasing the trapped droplet from an array is missing. Most of the applications that involve interaction between two different kinds of cells require incubation time that may vary from 2 to 24 hours. Such applications must trap the droplet containing interacting cells for a time duration and selectively release the droplet of interest. A microfluidic device with hundreds of docking sites has been shown to hold the droplets for hours^{43,44}; however, the ability to selectively release a droplet of interest has not been shown. Researchers have used passive methods, active methods, and a combination of both approaches to bring selectivity in droplet manipulation. However, active methods that use electrical, thermal, and magnetic actuation can have biocompatibility issues. Single-layer^{45,46} or multilayer valves^{47,48} can be combined with passive methods.

A breakthrough feature in the field would be to correlate functional traits with molecular signatures at single-cell resolution. This will lead to better understanding of drug effects and mechanisms of action, and will identify genetic markers and proteins that modulate increased immune cell activity.

### SUMMARY

The present technology provides devices and methods for single-cell analysis, isolation, and preparation for immunotherapy using a bottom-up approach. The technology allows functional phenotyping by observing cytotoxicity of cells and then probing the underlying biology. A droplet-based microfluidic device is capable of trapping droplets and release them selectively using microvalves. Each droplet can encapsulate natural killer cells (NK cells) and tumor cells for real-time monitoring of burst kinetics and spatial coordination during killing by single NK cells. Microvalve actuation is used to selectively release droplets with desired functional phenotypes, such as fast and serial killing of target tumor cells by NK cells. The technology allows for interrogating first interactions and real-time monitoring of kinetics and later cell recovery on demand for single-cell omic analysis, such as single-cell RNA sequencing (SCRNA).

In the microfluidic device, each droplet serves as an individual bioreactor that allows cell pairs to contact each other and form immunological complexes; all secreted molecules that promote paracrine signalling are retained within the microdroplet. The present technology has several advantages over bulk co-culture and other microfluidic approaches. These advantages include: (a) observations of immune cell motility, (b) quantification of single as well as serial interactions between the same cell pair; and (c) characterization of “one-to-one” interactions between two types of cells (e.g., NK and T cells) as well as “one-to-many” interactions, which can be studied in the same experiment by simply adjusting the initial cell density. The effector-to-target cell ratio can be established precisely. Multiple cell ratios can be observed in a single chip.

The present technology also can be summarized through the following list of features.

1. A microfluidic device for single cell analysis and isolation, the device comprising:
(a) an aqueous microdroplet generator comprising an oil inlet, one or more cell suspension inlets, and a flow focusing junction capable of forming a stream of aqueous microdroplets in the oil under continuous flow, the aqueous microdroplets containing the one or more cell suspensions;
(b) a flow layer comprising:
   (i) a flow channel, wherein the flow channel accepts the stream of aqueous microdroplets from the aqueous microdroplet generator at a first end and terminates in an outlet port at a second end;
   (ii) a plurality of docking stations for the aqueous microdroplets, the docking stations arranged in a plurality of parallel rows, each docking station comprising an entry port and an exit port, each docking station fluidically coupled to the flow channel through its entry port; and the docking stations configured for light microscopic observation; and
   (iii) a plurality of cell extraction channels, each cell extraction channel aligned in parallel to one of said rows of docking stations, each cell extraction channel coupled to each docking station of its aligned row of docking stations through the respective exit ports of the coupled docking stations, wherein each cell extraction channel terminates in first and second extraction outlets disposed at opposite ends of the cell extraction channel;
(c) a control layer comprising a plurality of parallel aligned control channels, each control channel comprising a plurality of microvalves and a pressure control port, wherein the control channels are aligned at right angles to said rows of docking stations, and wherein each microvalve is superimposed over one of said docking stations;
(d) a deformable membrane disposed between the flow layer and the control layer; wherein pressure of a fluid in a selected one of the control channels controls an activation state of the microvalves in said one of the control channels, and wherein release of an aqueous microdroplet from a docking site into the corresponding extraction channel is determined by the activation state of the microvalve overlapping the docking site and by a flow rate of oil in the flow channel.
   2. The microfluidic device of feature 1, wherein the aqueous microdroplet generator comprises two or more cell suspension inlets leading to a merging junction for mixing of two or more cell suspensions.
   3. The microfluidic device of feature 1 or feature 2, wherein the aqueous microdroplet generator is embedded within the flow layer.
   4. The microfluidic device of any of the preceding features, wherein the flow channel has a serpentine configuration having two or more linear sections arranged in parallel to one another, each linear section aligned with a row of docking stations, and wherein the linear sections are connected by curved sections of the flow channel not associated with docking stations.
   5 The microfluidic device of any of the preceding features, wherein the rows of docking stations contain from about 10 to about 100 docking stations per row.
   6. The microfluidic device of any of the preceding features, wherein the device contains from about 4 to about 20 rows of docking stations.
   7. The microfluidic device of any of the preceding features, wherein the device contains from about 40 to about 2000 docking stations.
   8. The microfluidic device of any of the preceding features, wherein the control channels and microvalves are configured for operation by a pressure-controlled gas introduced at the pressure control port.
   9 The microfluidic device of any of the preceding features, wherein each aqueous microdroplet in a docking station can be individually extracted by a combination of microvalve actuation and extraction channel flow.
   10. The microfluidic device of any of the preceding features, wherein the flow layer is housed in a first PDMS slab which is bonded to a glass substrate on one side and bonded on another side to a first side of the membrane, and wherein the control layer is housed in a second PDMS slab bonded to a second side of the membrane opposite to the first side.
   11. The microfluidic device of any of the preceding features, wherein the membrane comprises PDMS.
   12. The microfluidic device of any of the preceding features, wherein the membrane has a thickness of about 30-50 microns, such as about 40 microns
   13. The microfluidic device of any of the preceding features, wherein the aqueous microdroplet generator generates aqueous microdroplets having a diameter from about 150 microns to about 200 microns.
   14. The microfluidic device of any of the preceding features, wherein each microdroplet docking site has a diameter of about 200 microns.
   15. The microfluidic device of any of the preceding features, wherein the entry ports and exit ports of the aqueous microdroplet docking stations are configured to allow entry of aqueous microdroplets into all docking stations and their retention in the docking stations under a baseline oil flow condition, and to allow exit of an aqueous microdroplet only under higher oil flow induced by actuation of the microvalve overlapping the docking station housing the microdroplet.
   16. The microfluidic device of any of the preceding features, wherein the device is capable of isolating, analyzing, and delivering live individual cells of interest from a population of cells.
   17. A system for single cell analysis and isolation, the system comprising:
(a) the microfluidic device of any of the preceding features;
(b) fluid delivery devices to provide flow of oil into the oil entry port, one or more cell suspensions into the one or more cell suspension entry ports, and oil into the extraction channels;
(c) a microscope for observing cells in the aqueous microdroplets in the docking stations; and
(d) an imaging system for recording and analyzing images of cells obtained with the microscope.
   18. The system of feature 17, wherein the microscope is an inverted fluorescence microscope.
   19. The system of feature 17 or feature 18, wherein the system is capable of unattended, programmed analysis and extraction of cells of interest.
   20. The system of any of features 16-19, further comprising a cell culture system for culturing and/or expansion of cells isolated from aqueous microdroplets extracted from the microfluidic device.
   21. A method of single cell analysis and isolation, the method comprising the steps of:
(a) providing the microfluidic device of any of features 1-16, or the system of any of features 17-20;
(b) loading a plurality of individual cells into aqueous microdroplets in an oil stream using the microfluidic device;
(c) directing the microdroplets into the docking sites of the microfluidic device;
(d) incubating the docked microdroplets for a period of time and observing cell behaviour in the docked microdroplets, whereby individual microdroplets containing one or more cells of interest are identified; and
(e) extracting microdroplets containing one or more cells of interest by actuating one or more microvalves of the microfluidic device associated with docking stations containing the one or more cells of interest, and directing the extracted microdroplets to a collection device through one or more extraction outlets of the device.
   22. The method of feature 21, wherein step (e) comprises using an oil flow rate in the flow channel that is slow enough to allow cells to remain trapped within docking stations unless and until a microvalve is actuated.
   23. The method of feature 22, wherein actuation of a microvalve associated with a selected docking station causes an increase in oil flow rate through the docking station, whereby the aqueous microdroplet in the docking station moves out of the docking station through the docking station exit port and into an extraction channel.
   24. The method of any of features 21-23, wherein two different cell populations are mixed, and aqueous microdroplets are formed containing a mixture of the two different cell populations.
   25. The method of feature 24, wherein an interaction between cells from the two different cell populations are analyzed and the results used as a basis for selecting aqueous microdroplets for extraction.
   26. The method of feature 25, wherein the two different cell populations comprise immune effector cells and target cells of the immune effector cells.
   27. The method of feature 26, wherein the effector cells are natural killer (NK) cells and the target cells are cancer cells or cells infected with a microbial pathogen, such as a virus.
   28. The method of feature 26 or feature 27, wherein the effector cells and target cells are obtained from the same subject.
   29. A method of immunotherapy of a subject, the method comprising the steps of:
(a) providing the device of any of features 1-16, or the system of any of features 17-20 and samples of effector cells and target cells from the subject.
(b) loading the effector cells and target cells into docking stations of the device, whereby aqueous microdroplets are formed comprising both one or more effector cells and one or more target cells;
(c) observing behaviour of the cells for a period of time, whereby microdroplets are identified containing cells with desired actions by an effector cell against a target cell;
(d) extracting the identified microdroplets;
(e) isolating effector cells of interest from the extracted microdroplets;
(f) culturing the effector cells to expand their number; and
(f) administering the expanded effector cells to the subject.
   30. The method of feature 29, wherein the subject has cancer, the effector cells are NK cells of the subject, and the target cells are cancer cells of the subject.

### BRIEF DESCRIPTION OF DRAWINGS

FIG. 1 shows a schematic representation of a process for isolation and selective release of fast killing NK cells from a population of cells using a microfluidic device. The process includes the following steps: 1) generating aqueous microdroplets that co-encapsulate NK cells and tumor cells; 2) trapping the droplets in docking sites of the microfluidic device; 3) imaging the trapped droplets over a period of time, and thereby determining the killing activity of individual NK cells towards the tumor cells; 4) selectively releasing microdroplets containing fast killing NK cells; and 5) collecting cells of interest from the collected microdroplets. Collection of fast killing NK cells is depicted, but alternatively collection of killing-resistant tumor cells also can be performed, such as for genomic and/or proteomic analysis of the tumor cells.

FIGS. 2A-2D show schematic representations of a multilayered microfluidic device for trapping and selective release of aqueous microdroplets. FIG. 2A shows layer 1 (flow layer 100) of the microfluidic device with droplet generation section 150 capable of co-encapsulating NK cells and tumor cells in aqueous microdroplets, supplied through cell suspension inlets 110 and 115 and mixed at mixing junction 117, in an oil stream. Oil is introduced through oil inlet 105 into flow channel 130 and exits through oil outlet 120. In the embodiment depicted, the droplet trapping section consists of 4 rows of docking sites (a row of docking sites is also referred to as a docking site array 140), each row having two extraction outlets (145) and capable of holding 92 droplets at individual docking sites. FIG. 2B shows layer 2 (control layer 200) of the microfluidic device; layer 2 has microfluidic valves 230 that control the droplet docking sites of layer 1. The valves are disposed along control channel 220 at the sites of droplet junctions; control fluid is introduced at pressure control port 210. In FIG. 2C is depicted the combined assembly of layer 1, layer 2, and a deformable PDMS membrane between them. FIG. 2D shows a scaled up embodiment having 16 rows of docking stations, each row having 68 docking stations, for a total of 1088 docking stations for simultaneous monitoring and selectable extraction using a total of 1088 valves.

FIGS. 3A-3E show details of microfluidic valves. FIG. 3A is a light microscopic image showing the top view of a microfluidic device with valves located precisely overlaying droplet docking sites, with each row having two extraction outlets, one at each end of an extraction channel. FIG. 3B shows the resistive network of the first two docking sites, with each branch's hydrodynamic resistance represented as Rₙ₁, Rₙ₂ and Rₙ₃, where n represents the docking station number. The overall resistance of each docking site is represented as R₁ and R₂. FIG. 3C shows the resistive network of the first half of the row (11 docking sites) along with all hydrodynamic resistances and corresponding flow rates (Q).

FIG. 3D shows a light microscopic image of the top view of the docking site (left side), a schematic representation of a side view of the multilayered device (middle), and the flow rate and hydrodynamic resistance components when the valve is not actuated. There is no change in the control channel and flow channel volume in this case. In FIG. 3E the top view of the docking site, the side view of the device, and changes in flow rate and hydrodynamic resistance when the valve is actuated. It shows a localized increase in flow channel volume caused due to deformation of the thin membrane, which reduces the resistance and increases the flow rate locally.

FIG. 4 is a regime map showing trap and release modes of each section as a function of the continuous phase flow rate (μl per hour). Classification of row sections based on the location of docking sites is shown in the top part of the figure. The dotted line separates the regime of trap and release modes. Insets on the right and left sides of the figure show a microscopic images of a droplet trap and release in a particular section at the indicated flow rate of the continuous phase.

FIGS. 5A-5D show aspects of droplet release. FIG. 5A is a zoomed-in view of a docking site when the valve is actuated, showing L_{D} and L_{C}. FIG. 5B shows a characterization of the microfluidic system when the valve is fully actuated by plotting (L_{D}/L_{C}) ratio at constant (250 μl per hour) flow rate of the continuous phase plotted against the time required for release. The value of 1 represents droplet release, while ratio values between 0 and 1 represent the droplet still trapped. FIG. 5C presents a sequence of images showing selective droplet release from a docking site. The droplet gets trapped in a docking site when the valve is off. When the valve is actuated, the droplet starts moving out of the docking site through the connecting exit channel. Finally, the droplet comes out of the docking site. The neighbouring droplet trapped in the docking site is not released, showing how the valve can have selective release.

FIGS. 6A-6F show a variety of NK cell responses. FIG. 6A is a plot of the observed death time of individual K562 targets cells in droplets over 15 hours, divided into fast (less than 4 hours) average (4-12 hours) and slow (greater than 12 hours). N values are; fast killing: 18 cells, average killing: 40 cells, slow killing: 7 cells (target cells that survived the 15 hours not represented). FIG. 6B shows an image sequence displaying a single droplet containing one live slow killing NK92 and K562 cell; the K562 tumour cell is labelled green, and the NK92 is unlabelled. The K562 is alive at 9 hours.

FIG. 6C shows an image sequence displaying a single droplet containing one live average killing NK92 and a K562 cell. The process of cell death starts after 6 hours. FIG. 6D shows an image sequence displaying a single droplet containing one live NK92 and a K562 cell. The K562 cell dies after 3 hours.

FIG. 6E shows an image sequence displaying a single droplet containing one live NK92 and two K562 cells. Both the cells die at 15 hours. (1) Image sequence displaying the selective release of the droplet of interest (fast killing NK cell) shortly after target cell death.

### DETAILED DESCRIPTION

A droplet-based microfluidic platform technology has been developed that possesses integrated multilayer microvalves to trap and selectively release a microdroplet of interest containing encapsulated cells or pairs or groups of cells, such as interacting cells. The technology can be used for selectively releasing droplets of interest, and the particular cells they contain, from an array of droplets. A flow-focusing device generates water-in-oil droplets, and the aqueous phase contains cells of interest, which can be a mixture of cells whose interaction is then observed in an array of docked microdroplets simultaneously. The microvalves in a different layer are precisely placed over the docking sites, and a deformable membrane separates these layers. Cells with desired phenotypes can be isolated and recovered for analysis or expansion and use in cell-based therapy.

Each docking site can be monitored over a period of hours to identify droplets containing desired cells. The system is biocompatible, making it ideal for single-cell analysis, drug screening, and interdisciplinary research applications. Upon observing a particular event, the ability to immediately release droplets of interest allows for recollection of viable effector cells, which is essential for certain types of downstream analysis such as transcriptomic sequencing. The present microfluidic system can trap and release a droplet from an array of up to thousands of trapped droplets, with selective release of individual droplets. The system can be applied to a wide range of different phenotypic markers of interest, including motility, calcium signalling, serial killing, viability, secretions, and other features that can be observed using microscopy, including fluorescence microscopy. A more precise correlation between-genomics and proteomics of immune cells, leading to more successful immunotherapy.

The present technology integrates a layer of pressure-actuated microvalves into a microfluidic device for formation and visualization of aqueous microdroplets containing individual cells. The new device is capable of selective collection of droplets containing the cells having desirable functional properties. Collected cells can be subjected to any form of analysis, including scRNA analyses and other features related to immune cell activity. Thus, the technology utilizes a visual representation of cellular activity, such as killing kinetics of NK cells, cell-cell interactions, or various behaviors observed via time-lapse imaging to extract droplets of interest. Unlike flow-cytometry based sorting and other methods with no visual component, the present technology can be used to select cells based on precise criteria for release, analysis, or scale-up. With time-lapse microscopy, the user can observe a wide variety of cell behaviours that might warrant further biological analysis to uncover underlying mechanisms. With techniques such as sequencing and mass spectrometry, genomic and proteomic differences can be observed using populations only tens of cells per condition. In immunotherapy, uncovering the biological mechanisms involved in effective versus ineffective immune cells can be utilized to greatly enhance the quality of therapy by selective cell screening or treatment to upregulate certain factors.

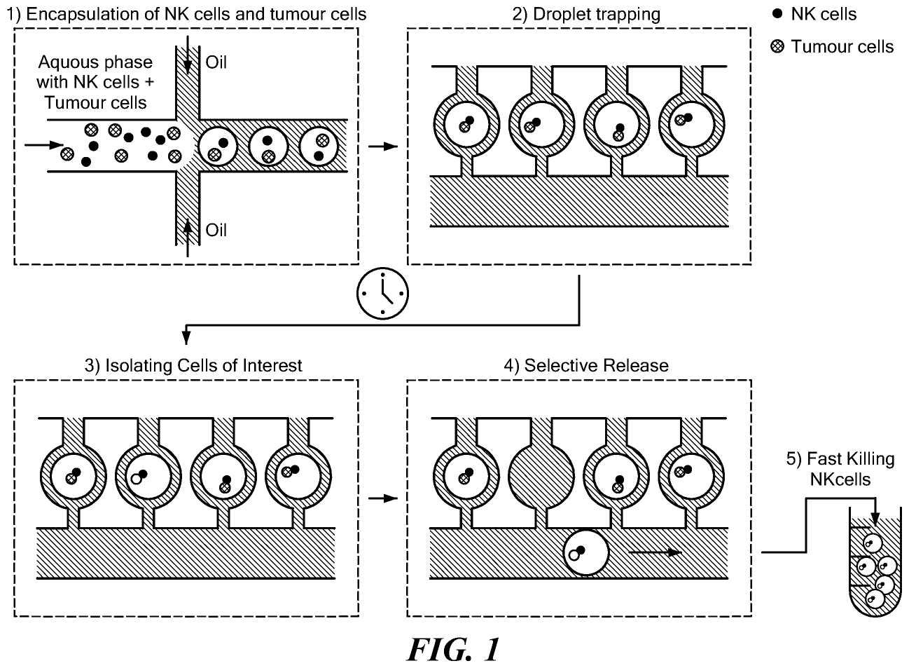

In an example of the present technology, a microfluidic device could selectively release a droplet containing NK92 cells and one or more K562 leukemia cells line using a multilayer valve and the process shown schematically in FIG. 1. The NK92 cell line was chosen for the high cytotoxicity of the cells and ease of culturing. K562 was chosen as a target cell due to its high susceptibility to NK cells and standard use as markers of NK cell cytotoxic potential⁵³⁻⁵⁵. The device contained three layers: a flow layer, a control layer and a deformable membrane disposed between the flow and control layers. When the valve is off, droplets are trapped in the docking sites, and when the valve is turned on, droplets are selectively released. For these experiments, the time required for NK cells to kill target cells was utilized as a simple but effective indicator of NK cell cytotoxic potential. The NK cells were allowed to interact with tumour cells and imaged every 15 minutes, keeping the valve off. The NK cells that showed fast killing ability were then selectively released from the docking sites with the actuation of the valve. The cells collected could be subjected to single-cell sequencing or mass spectrometry to uncover the underlying physiologic producing the observed characteristics. As an option, a microcontroller-based control system (e.g. Arduino) can be assembled to allow easy and user-friendly valve actuation control and a scaled-up design to allow a significant number of cells to be isolated in one operation.

### Device Design and Operation

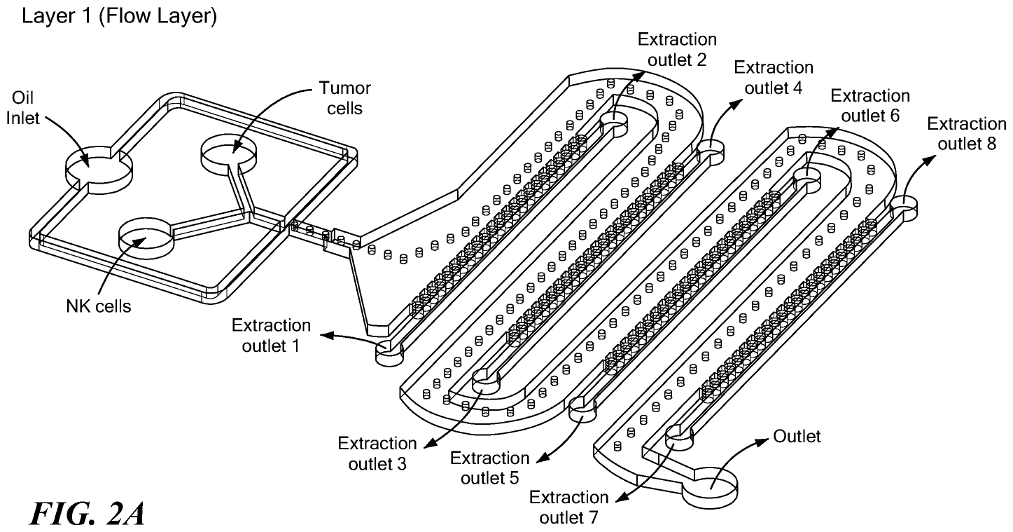

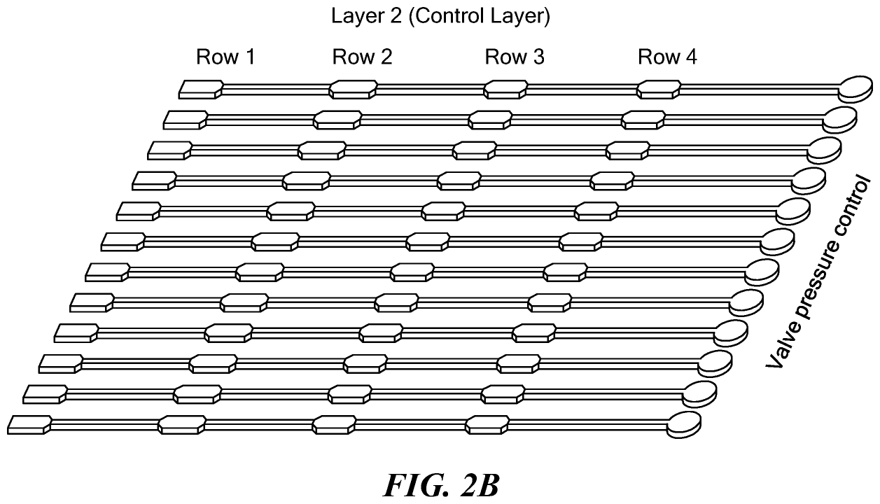

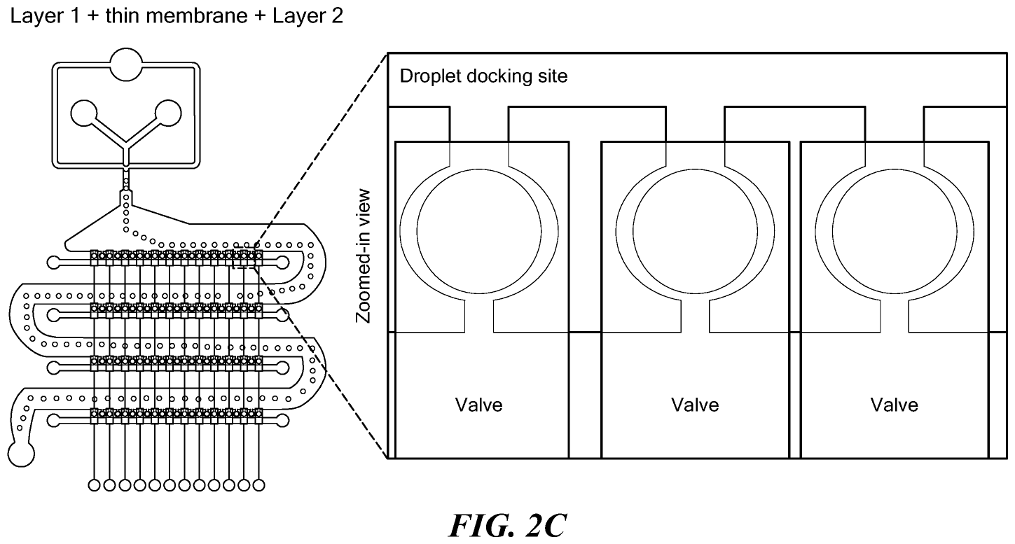

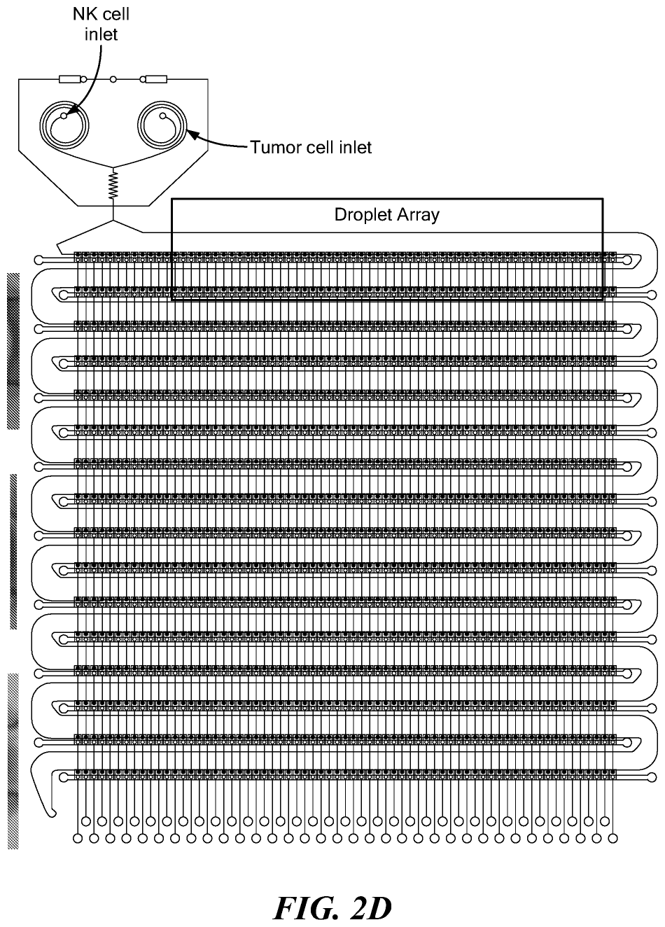

The schematic of a microfluidic device is shown in FIGS. 2A-2C. The device includes a flow layer (layer 1), a control layer (layer 2), and a deformable thin membrane disposed between layers 1 and 2. The flow layer contains a droplet generation section and a droplet trapping section. See FIG. 2A. The droplet generation section uses flow-focusing geometry; i.e., interaction between an oil phase and an aqueous phase containing a mixture of two cell types (e.g., NK cells and target cells), to generate aqueous microdroplets, which can have a diameter ranging from 150 μm to 200 μm, for example. The droplet trapping section contains docking sites; each docking site can have a diameter of about 200 μm, for example, and is capable of entrapping an aqueous microdroplet under flow of an oil stream containing the microdroplet. The control layer contains valves and has rows and columns selected to align the valves with the docking station, one valve aligning with each docking station of an array. See FIG. 2B, depicting the control layer, and FIG. 2C, depicting the docking station with microdroplet and inlet and exit ports, and overlapping valve. For example, there can be four rows and twenty-three columns in an array of docking stations on a microfluidic chip (see FIG. 2A).

The air inlets of the control layer can be used to control each column of the array. By opening or closing the extraction outlet of a selected row (when open, oil is flowed through the extraction channel to collect selected and extracted aqueous microdroplets in the extraction oil stream) simultaneously with adjusting the airflow of a selected column of the control layer, each valve can be selectively controlled precisely and individually. The membrane separating the flow and control layers is a thin deformable membrane, for example a polymer membrane of 40 μm thickness. The thickness and material of the membrane are selected such that the membrane can be deformed under the action of air flow in the control channel, with the result that the membrane is deformed when reduced pressure is applied in a valve, thereby enlarging the flow channel beneath the valve and selectively increasing the flow rate through the corresponding docking station, forcing the microdroplet sequestered in the docking station outward through an exit port of the docking station and into a collection channel.

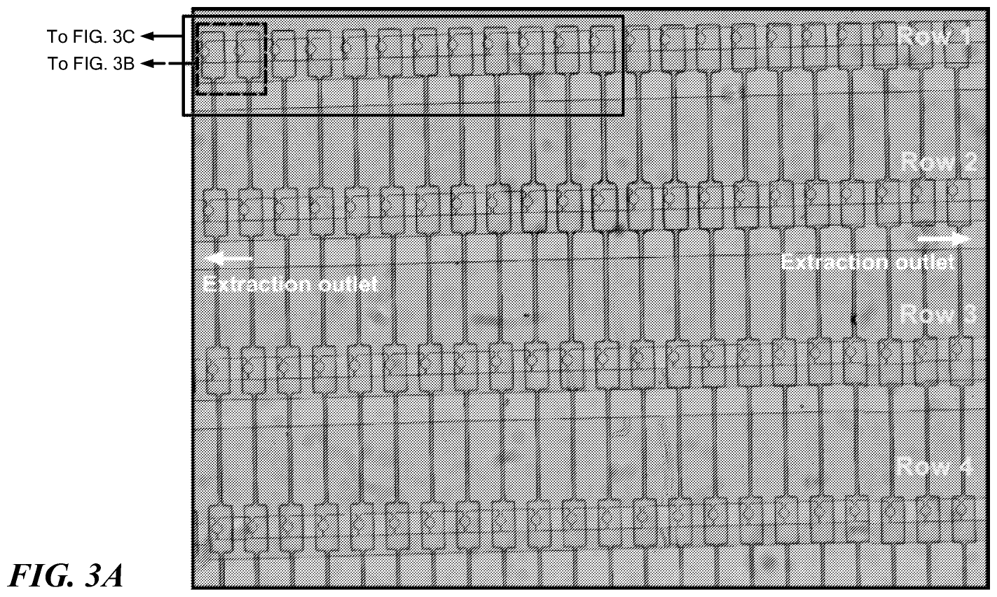

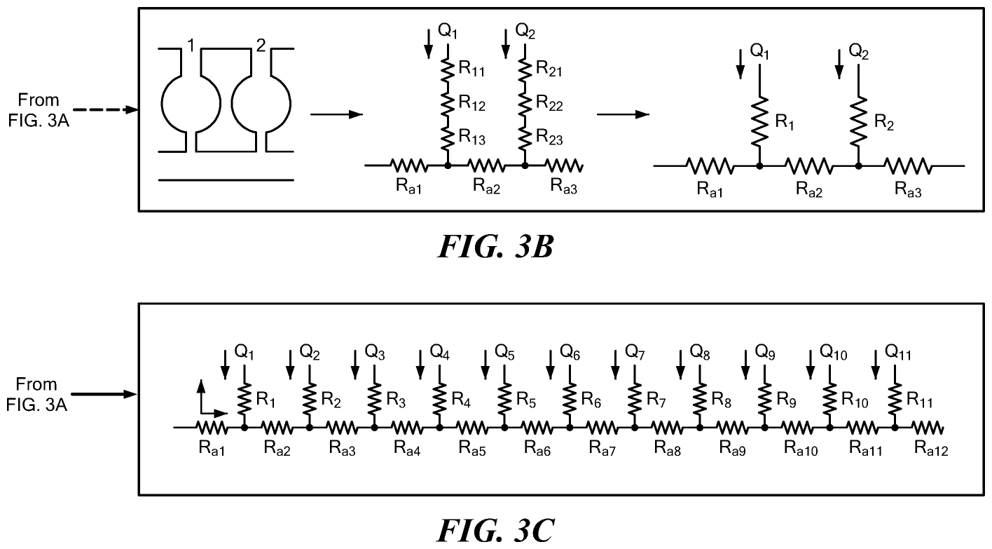

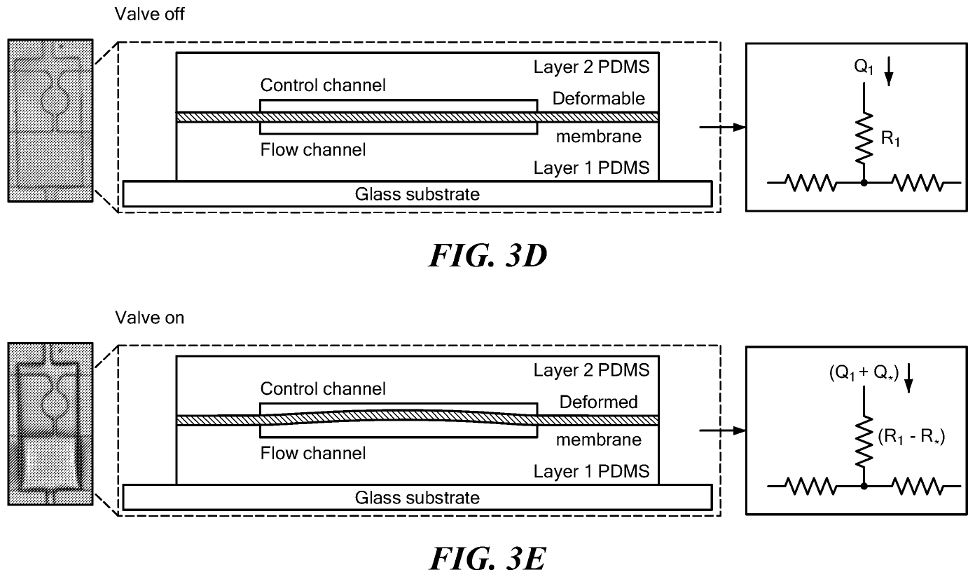

FIGS. 3A-3E show the overall device design, hydrodynamic resistive network, and device operation. FIG. 3A shows a light microscope image of the top view of an entire device with a valve at each docking site. The trapping and releasing of droplets from each docking site are governed by the hydrodynamic resistance from the docking site to the extraction outlet. The entire microfluidic system can be considered similar to an electric resistive network, where flow rate (Q) is analogous to the current, electric resistance is analogous to the hydrodynamic resistance (R), and pressure drop (ΔP) is equivalent to the potential difference. The hydrodynamic resistance in each branch can be calculated using the equation below,

$$R = \frac{12 μ l}{{wh}^{3} [ 1 - 0.63 ( h / w ) ]}$$

where w, h, l, and μ represent the width, height, length, and viscosity of continuous phase, respectively. FIG. 3B shows the effective hydrodynamic resistance in each part of the docking site. R₁₁, R₁₂ and R₁₃ represent the hydrodynamic resistance of entry port 142, docking site 141, and exit port 143 of the first docking site, respectively. The overall resistance of the first docking site is R₁=R₁₁+R₁₂+R₁₃, similarly for the second (R₂), third (R₃), fourth (R₄) docking sites. In the same way, Q₁, Q₂, Q₃ . . . represent the flow rate entering the first, second, and third docking sites. FIG. 3C shows the resistive network of the half row (11 docking sites). Here only half of the row is considered because there are two extraction outlets in each row, located at the ends of extraction channel 144. Each half of the row has similar hydrodynamic resistance to the other half; hence each half row is symmetrically equivalent to the other half. The effective resistance between the entry port of the docking site to the extraction channel is R=R₁+Rₐ₁; similarly for the second docking site R=R₁+Rₐ₁+Rₐ₂. As effective resistance of the first docking site is always less than the next docking site, hence flow rate entering the first docking site is always more than the other docking sites (Q₁>Q₂>Q₃> . . . . Q₁₁).

Using the resistive network described above, trapping and releasing a droplet can be explained. For trapping a droplet, the effective resistance from the entry of the docking site to the extraction outlet is the most critical parameter. The droplet takes the path that offers the least hydrodynamic resistance. Hence, the droplet occupies and gets trapped in the first docking site with the least effective hydrodynamic resistance (R₁+Rₐ₁). Once the first docking site is filled with the droplet, its resistance increases rapidly due to blocking by the droplet; the least resistant path for the next droplet then becomes the second docking site (R₁+Rₐ₁+Rₐ₂). Overall, docking sites closer to the extraction outlets get filled first with droplets, while docking sites furthest away from the extraction outlets get filled last. Once all the docking sites are filled, any additional droplets go to a waste outlet provided on the chip.

Similarly, for selectively releasing the trapped droplet from a docking site, it is essential to reduce the hydrodynamic resistance locally and thereby increase the flow rate (Q) entering the particular docking site. FIGS. 3D and 3E show the valve located over one of the docking sites (micrograph at left side), a side view of the device when the valve is not actuated (center portion of FIG. 3D) or actuated (center portion of FIG. 3E) and the corresponding hydrodynamic resistances (right side of FIGS. 3D and 3E). In the case of valve actuation, the thin membrane is deformed as shown in FIG. 3E, bowing outwards into the control channel at the valve location. A suction effect on the membrane from the control channel side of the valve causes a deformation of the thin membrane, which causes a localized increase in the flow channel volume. The localized increase in the volume of the flow channel reduces the hydrodynamic resistance of the corresponding docking site. The corresponding decrease in hydrodynamic resistance is given by (R₁−R*), where R* is the reduction in hydrodynamic resistance due to an increase in volume. Hence effective resistance for trapping/releasing the droplet from the first docking site becomes R=(R₁−R*)+Rₐ₁. The reduction in hydrodynamic resistance causes a localized flow rate increase in the docking site, given as Q₁+Q*, where Q* represents an increase in flow rate.

### EXAMPLES

### Example 1. Microfluidic Device Fabrication

The microfluidic device flow channel (layer 1) and control channel (layer 2) were fabricated using standard soft lithography. The photomasks required for the flow channel and control channel were drawn in AutoCAD. Negative photoresist SU8 was spin-coated on a 4-inch silicon wafer and subjected to UV light for preparing the master mold. Polydimethylsiloxane (PDMS) and curing agent were mixed in a ratio of (10:1) and poured on the master mold of both layers to prepare a replica in PDMS. The thin deformable PDMS membrane was prepared by spin coating PDMS+curing agent (10:1) at 1000 rpm for 2 minutes. Finally, all three layers were assembled: The control layer was punched for an air inlet and bonded to the PDMS membrane by subjecting it to air plasma (Harrick Plasma). The control layer and PDMS membrane were bonded with the flow channel by precisely aligning valves over docking sites under the microscope. This entire assembly was then bonded to a glass slide. One-hour baking followed each bonding process in a convection oven at 70° C.

### Example 2. Cell Culture

NK92 cells and K562 human leukemia cells were acquired from ATCC. NK-92 were cultured using X-VIVO10 medium (Lonza) supplemented with 10% heat-inactivated FBS (Gibco) and 500 IU/mL IL2 (Prospec). K562 were cultured in RPMI1640 medium supplemented with 10% heat-inactivated FBS and 1% antibiotic-antimycotic mixture (Gibco). Cell media containing cells in suspension were combined at a 1:1 ratio for droplet co-encapsulation. Cells were kept at 37° C. and 5% CO₂. Both NK92 cells and K562 human leukaemia cells were used at a concentration of 3×10⁶ cells/mL.

### Example 3. Aqueous Microdroplet Generation

The water-in-oil droplets were generated upstream using a flow-focusing junction. The aqueous phase consisted of NK cells and tumour cells mixed before reaching a flow-focusing junction at 250 μl per hour, as shown in FIG. 2A. FC-40 with 2% Span-80 surfactant was used as the oil phase with continuous flow at 1000 μl per hour. The oil and aqueous phases were pumped into the microfluidic device using three syringe pumps. Under the flow-focusing geometry dimensions, droplets having a diameter in the range of 150 to 200 μm were generated. These generated droplets were trapped in docking sites of the device; all the outlets were kept open during the droplet generation and docking process. Once all the docking sites were filled, aqueous phase flow was stopped, while oil was allowed to flow, resulting in no droplet generation. The droplets encapsulating NK cells and tumor cells trapped in a docking site were incubated at 37° C. in a microscope chamber for imaging live experiments to observe the killing activity of NK cells.

### Example 4. Microscopy

Device images and data were acquired using a Zeiss Axio Observer.Z1 with Hamamatsu C10600 Orca-R2 digital camera. Fluorescence was observed using standard FITC and dsRed filters. The microscope was equipped with an incubated stage to maintain cells at 37° C. and 5% CO₂ during experiments. Target cell killing was observed at 20× magnification every 15 minutes over the course of 15 hours. Results were analyzed using Zen Blue software (Zeiss). Droplet release was recorded at 5× magnification using bright field imaging. Images of the entire device array were taken at 10× magnification and processed using the Zen Blue stitching feature.

### Example 5. Regimes of Droplet Trap and Release Without Valve Actuation

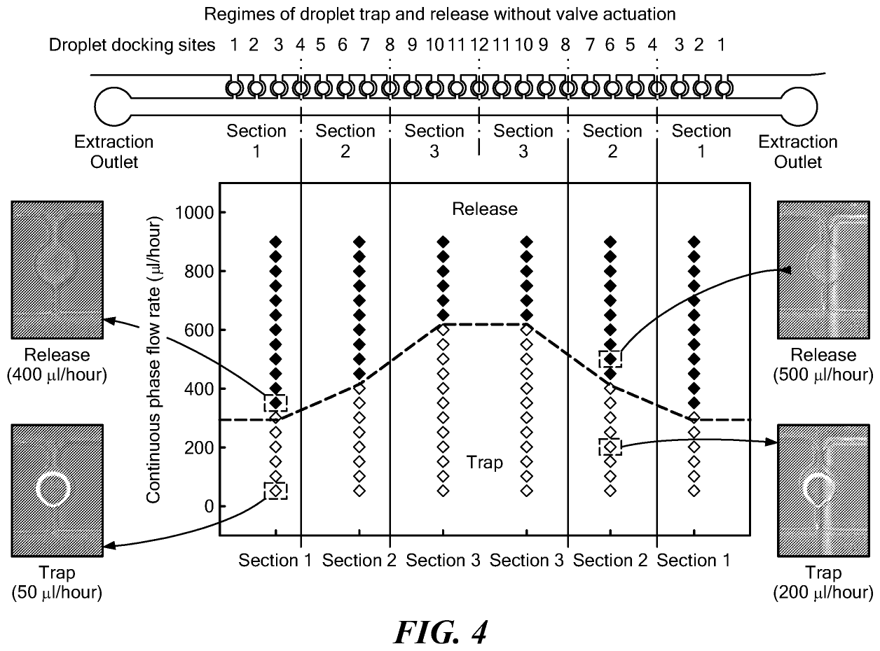

Once droplets were trapped in docking sites, only a continuous phase (oil) was allowed to flow, stopping the droplet generation. The continuous phase flow rate was then systematically increased from 0 to 900 μl per hour to investigate regimes of droplet trapping and release without actuation of the valve. Furthermore, only extraction outlets in a row of interest and the main outlet were kept open to the atmosphere, while other extraction outlets were closed. It was observed that under this condition, each row behaved similarly. Further, it was observed that droplets trapped in a docking site closer to an extraction outlet were more easily released with an increase in oil flow rate than the droplets trapped in docking sites further away from an extraction outlet. As a result, each row of docking sites can be divided into three sections: section 1, section 2 and section 3, with section 1 being closest to the extraction outlet on both sides and section 3 being farthest. The number of sections was arbitrarily chosen as 3; other numbers of sections could be selected, and would reveal the same trend. It was observed that for section 1, once the flow rate of oil exceeded 300 μl per hour, the droplet was released without actuation of the valve. The exact threshold for this phenomenon will depend on the design and resistances of the particular microfluidic device. Similarly, for sections 2 and 3, the threshold values for microdroplet release without valve actuation was 400 and 600 μl per hour, respectively, as shown in FIG. 4. The dotted line in FIG. 4 separates the regime of droplet trapping and release by plotting each section against the continuous phase flow rate. The regime map is helpful in the operation of the system. In the microfluidic device embodiment tested, the continuous phase flow rate had to be lower than 300 μl per hour to keep all the droplets in their docking sites.

### Example 6. Selective Droplet Release With Valve Actuation

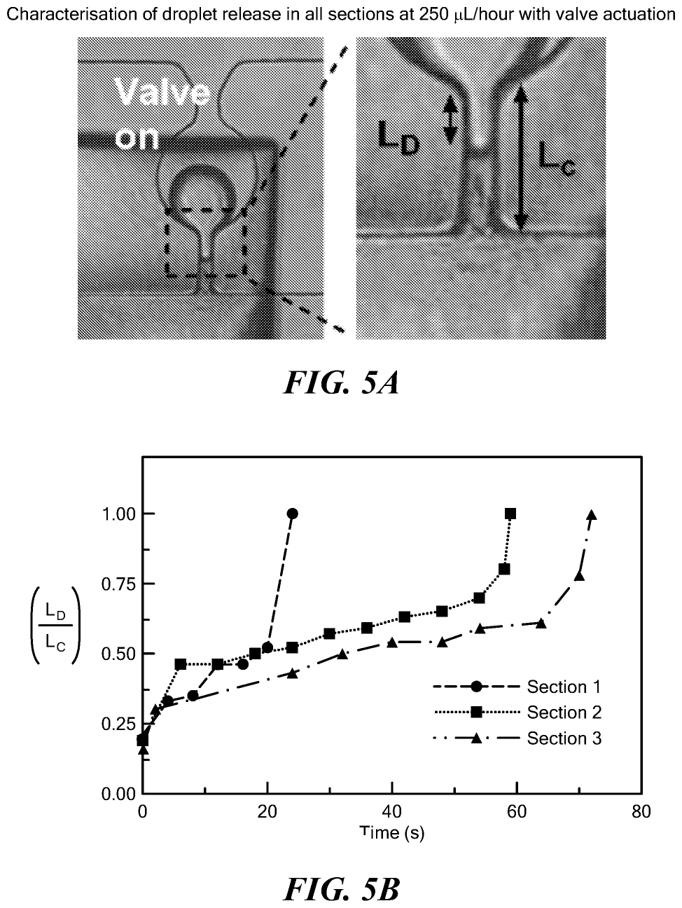

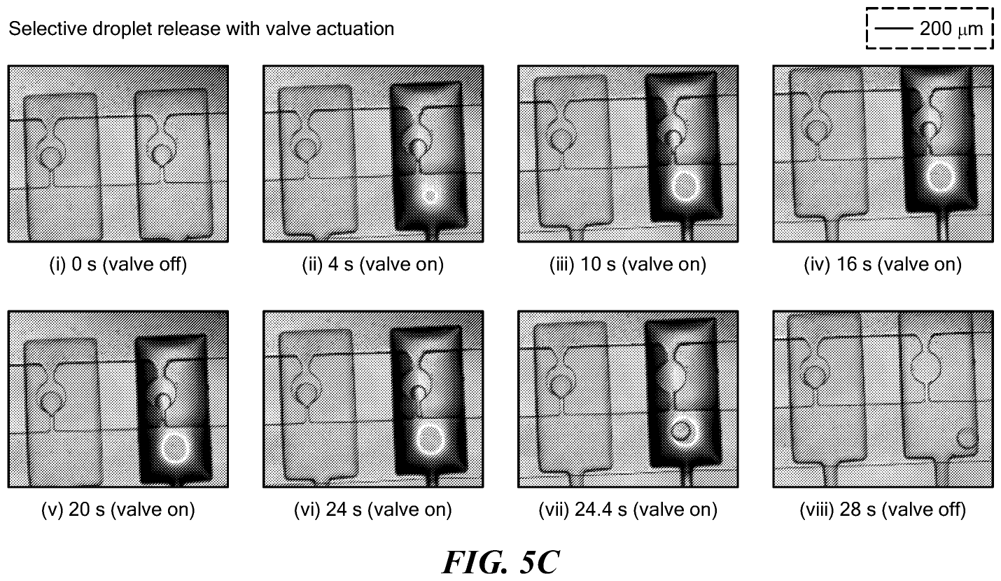

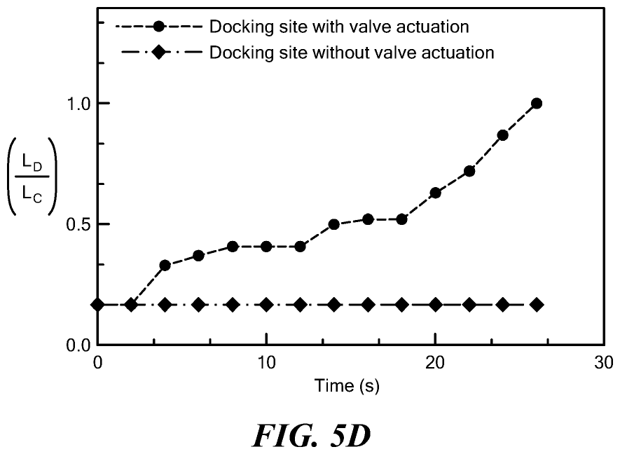

Next, the valve in the regime of trapping was actuated to characterize the microfluidic system. The L_{D}/L_{C} ratio was plotted against the time required for droplet release when the valve was actuated for each section while keeping the continuous oil phase flow rate constant at 250 μl/hour. L_{D} represents the droplet length entering a connecting exit channel, and L_{C} is the length of the connecting exit channel shown in FIG. 5A. The ratio of L_{D}/L_{C} represents a process of droplet release once the valve was actuated. The value L_{D}/L_{C}=1 represents the droplet released from the docking site, while a value between 0 and 1 represents the droplet still trapped in a docking site. It was observed that, for section 1, the droplet was released more quickly than for section 2 and section 3, as shown in FIG. 5B.

After characterization of the system for droplet release, the selective release of a droplet from a docking site was demonstrated. The droplet was trapped in a docking site of section 1 in the first row to demonstrate selective droplet release. The continuous phase flow rate was kept constant at 250 μl per hour, which kept the droplet trapped in the docking site. An image sequence of selective droplet release is shown in FIG. 5C. FIG. 5C shows two droplets trapped in two adjacent docking sites. Both of the docking sites had the valve precisely placed just over the docking site. Initially, both the valves were off, keeping the droplets trapped in docking sites. When the valve on the right side was turned on, there was a localized increase in volume at the docking site, which caused the droplet to start moving into the connecting channel exit of the docking site, increasing the ratio of L_{D}/L_{C} with time, as shown in FIG. 5D. At 24.4 s, the droplet exited the docking site into an extraction channel that connects with the extraction outlet. Later (time 28 s), the droplet moved towards the extraction outlet.

The docking site with the actuated valve did not affect the adjoining docking site, as visible in the image sequence of FIG. 5C. The plot of L_{D}/L_{C} for the droplet trapped in the docking site without valve actuation is shown in FIG. 5D. There was no change in L_{D}/L_{C} for the droplet trapped in the docking site without a valve actuation with time. Thus, selective release of the droplet with valve actuation has been demonstrated, proving that the valve has a very localized effect.

### Example 7. Isolation of Droplets Containing Cytotoxic NK Cells

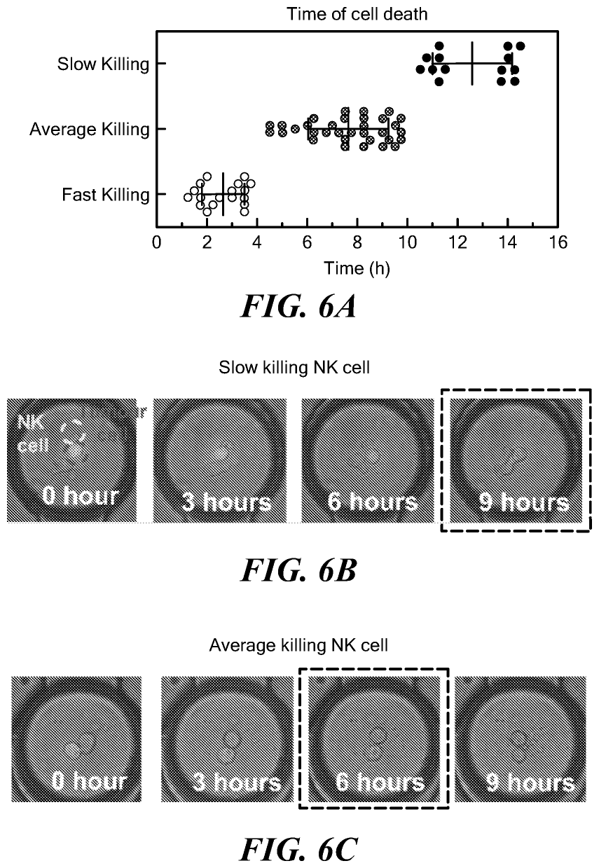

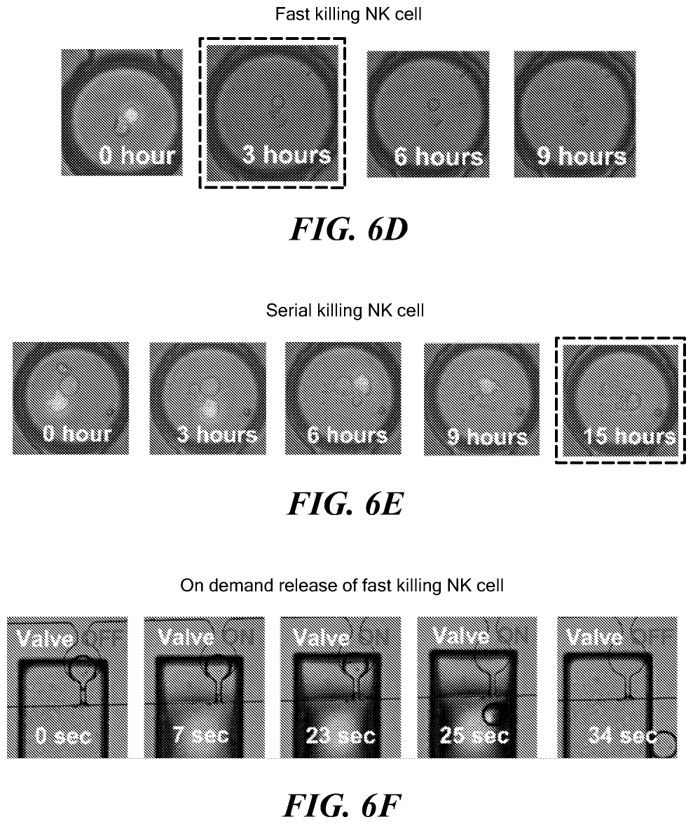

To determine the standard kinetics for NK92 cells to kill K562 cancer cells using the present technology, droplet co-encapsulations of these two cell types were monitored over the course of 15 hours. Calcein AM (green) and ethidium homodimer (red) were included to help determine target cell death. Cell deaths were discernible by an abrupt morphological change, accompanied by abrupt secretion of calcein from the cell and later an accumulation of ethidium in the nucleus. A total of 89 co-encapsulations were observed, and of those, 65 resulted in the killing of target K562 cells. Based on the average times of target cell death, the NK cells were split into three groups: fast killing, average killing and slow killing (FIG. 6A). Fast killing NK cells (20% of observed cells) were able to kill their targets within 4 hours, while slow killing NK cells (43% of observed cells) took more than 10 hours to kill or did not kill at all during the 15-hour experiment. The remaining NK (37% of observed cells) killed their targets at some point between 4 and 12 hours of co-encapsulation

FIG. 6B shows an image sequence depicting a droplet containing a single NK92 and a single K562 cell. The K562 cell was still intact and viable at 9 hours; hence, it was a slow killing NK92. Similarly, FIGS. 6C and 6D show image sequences showing killing activity of average and fast killing NK92 cells. For an average killing NK92 cell, target cell death starts at 6 hours, and the K562 died before 9 hours had elapsed. A loss of green fluorescence accompanied by a morphological change of the K562, indicating cell death, was observed. In the case of a fast killing NK92 cell, target cell death was observed at 3 hours, at which time a considerable change in fluorescence was observed compared to that at 0 hours, as shown in FIG. 6D. Finally, the serial killing ability of an NK92 is shown in FIG. 6E, where one NK92 killed two K562 cells by 15 hours.

Based on these criteria, the identification and collection of NK cells that could kill within 4 hours or less was performed. Shortly after the killing event was observed, the droplet was selectively released from the device, and the NK cell was recollected (FIG. 6F). Notably, this allowed collection of the NK cells while they were still alive and active. This demonstrates the ability of the present technology to identify effector cells of interest and quickly collect them while the cells are still intact and viable. While this is not necessary for single-cell downstream analytical techniques, isolation of viable cells can be valuable for subsequent analysis or cell expansion. For methods such as sequencing, droplets can be directly collected into lysis buffer to preserve integrity of nucleic acids. To validate cell survival of the droplet selection process, the cell viability was confirmed for one hour after droplet isolation in the collection channel. Using calcein-AM, it was confirmed that the collection process did not appear to negatively impact the viability of NK92 cells or K562 cells.

### REFERENCES

1 M. W. Rohaan, S. Wilgenhof and J. B. A. G. Haanen, Virchows Arch., 2019, 474, 449-461.
2 L. Zhang, M. Liu, S. Yang, J. Wang, X. Feng and Z. Han, Am. J. Cancer Res., 2021, 11, 1770-1791.
3 H. E. Ghoneim, A. E. Zamora, P. G. Thomas and B. A. Youngblood, Trends Mol. Med., 2016, 22, 1000-1011.
4 D. Di, P. Oberoi, K. Kamenjarin, J. Francisco, V. Ossa, B. Uherek, H. Bönig and W. S. Wels, Cells, 2020, 9, 1-20.
5 M. Granzin, J. Wagner, U. Köhl, A. Cerwenka, V. Huppert and E. Ullrich, Front. Immunol., DOI: 10.3389/fimmu.2017.00458.
6 Emily M. Mace and Jordan S. Orange, Immunobiology, 2011, 118, 2363-2364.
7 J. Xie, C. M. Tato and M. M. Davis, Immunol. Rev., 2013, 251, 65-79.
8 S. Darmanis, C. J. Gallant, V. D. Marinescu, M. Niklasson, A. Segerman, G. Flamourakis, S. Fredriksson, E. Assarsson, M. Lundberg, S. Nelander, B. Westermark and U. Landegren, Cell Rep., 2016, 14, 380-389.
9 K. Guldevall, B. Vanherberghen, T. Frisk, J. Hurtig, A. E. Christakou, O. Manneberg, S. Lindström, H. Andersson-Svahn, M. Wiklund and B. Önfelt, PLOS One, DOI: 10.1371/journal.pone.0015453.
10 Y. J. Yamanaka, C. T. Berger, M. Sips, P. C. Cheney, G. Alter and J. C. Love, Integr. Biol. (United Kingdom), 2012, 4, 1175-1184.
11 P. J. Lee, P. J. Hung, R. Shaw, L. Jan and L. P. Lee, Appl. Phys. Lett., 2005, 86, 1-3.
12 A. M. Skelley, O. Kirak, H. Suh, R. Jaenisch and J. Voldman, Nat. Methods, 2009, 6, 147-152.
13 B. Dura, Y. Liu and J. Voldman, Lab Chip, 2014, 14, 2783-2790.
14 S. Faley, K. Seale, J. Hughey, D. K. Schaffer, S. Vancompernolle, B. Mckinney, F. Baudenbacher, D. Unutmaz and J. P. Wikswo, Lab Chip, 2008, 8, 1700-1712.
15 J. P. Frimat, M. Becker, Y. Y. Chiang, U. Marggraf, D. Janasek, J. G. Hengstler, J. Franzke and J. West, Lab Chip, 2011, 11, 231-237.
16 T. P. Lagus and J. F. Edd, J. Phys. D. Appl. Phys., DOI: 10.1088/0022-3727/46/11/114005.
17 I. K. Dimov, R. Lu, E. P. Lee, J. Seita, D. Sahoo, S. M. Park, I. L. Weissman and L. P. Lee, Nat. Commun., DOI: 10.1038/ncomms4451.
18 B. Dura, S. K. Dougan, M. Barisa, M. M. Hoehl, C. T. Lo, H. L. Ploegh and J. Voldman, Nat. Commun., DOI: 10.1038/ncomms6940.
19 K. Chung, C. A. Rivet, M. L. Kemp and H. Lu, Anal. Chem., 2011, 83, 7044-7052.
20 H. Yin and D. Marshall, Curr. Opin. Biotechnol., 2012, 23, 110-119.
21 A. E. Sgro, P. B. Allen and D. T. Chiu, Anal. Chem., 2007, 79, 4845-4851.
22 E. X. Ng, M. A. Miller, T. Jing and C. H. Chen, Biosens. Bioelectron., 2016, 81, 408-414.
23 T. Konry, S. Sarkar, P. Sabhachandani and N. Cohen, Annu. Rev. Biomed. Eng., 2016, 18, 259-284.
24 M. R. Raveshi, M. S. Abdul Halim, S. N. Agnihotri, M. K. O'Bryan, A. Neild and R. Nosrati, Nat. Commun., DOI: 10.1038/s41467-021-23773-x.
25 P. Garstecki, M. J. Fuerstman, H. A. Stone and G. M. Whitesides, Lab Chip, 2006, 6, 437-446.
26 S. L. Anna, N. Bontoux and H. A. Stone, Appl. Phys. Lett., 2003, 82, 364-366.
27 C. Cramer, P. Fischer and E. J. Windhab, Chem. Eng. Sci., 2004, 59, 3045-3058.
28 D. R. Link, S. L. Anna, D. A. Weitz and H. A. Stone, Phys. Rev. Lett., 2004, 92, 054503.
29 L. Ménétrier-Deremble and P. Tabeling, Phys. Rev. E, 2006, 74, 035303 (R).
30 Z. Che, N. T. Nguyen and T. N. Wong, Appl. Phys. Lett., 2011, 98, 2009-2012.
31 L. Xu, H. Lee, R. Panchapakesan and K. W. Oh, Lab Chip, 2012, 12, 3936.
32 H. D. Xi, H. Zheng, W. Guo, A. M. Gañán-Calvo, Y. Ai, C. W. Tsao, J. Zhou, W. Li, Y. Huang, N. T. Nguyen and S. H. Tan, Lab Chip, 2017, 17, 751-771.
33 A. R. Abate, J. J. Agresti and D. A. Weitz, Appl. Phys. Lett., DOI: 10.1063/1.3431281.
34 A. Huebner, D. Bratton, G. Whyte, M. Yang, A. J. Demello, C. Abell and F. Hollfelder, Lab Chip, 2009, 9, 692-698.
35 W. Wang, C. Yang and C. M. Li, Lab Chip, 2009, 9, 1504-1506.
36 W. Wang, C. Yang, Y. Liu and C. M. Li, Lab Chip, 2010, 10, 559-562.
37 X. Niu, S. Gulati, J. B. Edel and A. J. Demello, Lab Chip, 2008, 8, 1837-1841.
38 X. Chen, A. Brukson and C. L. Ren, Microfluid. Nanofluidics, 2017, 21, 1-10.
39 D. J. Collins, T. Alan, K. Helmerson and A. Neild, Lab Chip, 2013, 13, 3225-3231.
40 M. R. Raveshi, S. N. Agnihotri, M. Sesen, R. Bhardwaj and A. Neild, Sensors Actuators B. Chem., 2019, 292, 233-240.
41 S. N. Agnihotri, M. R. Raveshi, R. Bhardwaj and A. Neild, Microfluid. Nanofluidics, 2021, 25, 1-13.
42 C. N. Baroud, J. P. Delville, F. Gallaire and R. Wunenburger, Phys. Rev. E-Stat. Nonlinear, Soft Matter Phys., 2007, 75, 1-5.
43 S. Sarkar, P. Sabhachandani, D. Stroopinsky, K. Palmer, N. Cohen, J. Rosenblatt, D. Avigan and T. Konry, Biomicrofluidics, 2016, 10, 1-12.
44 S. Sarkar, N. Cohen, P. Sabhachandani and T. Konry, Lab Chip, 2015, 15, 4441-4450.
45 S. N. Agnihotri, M. R. Raveshi, R. Bhardwaj and A. Neild, Langmuir, 2020, 36, 1138-1146.
46 S. N. Agnihotri, M. R. Raveshi, R. Bhardwaj and A. Neild, Phys. Rev. Appl., 2019, 11, 034020.
47 M. A. Unger, H. P. Chou, T. Thorsen, A. Scherer and S. R. Quake, Science (80-.), 2000, 288, 113-116.
48 H. W. Zhu, N. G. Zhang, R. X. He, S. Z. Li, S. S. Guo, W. Liu and X. Z. Zhao, Microfluid. Nanofluidics, 2011, 10, 1343-1349.
49 G. D. Evrony, A. G. Hinch and C. Luo, Annu. Rev. Genomics Hum. Genet., 2021, 22, 171-197.
50 A. Kulkarni, A. G. Anderson, D. P. Merullo and G. Konopka, Curr. Opin. Biotechnol., 2019, 58, 129-136.
51 L. M. Hu R, Li Y, Yang Y, Mass Spectrom Rev.
52 P. Hu, W. Zhang, H. Xin and G. Deng, Front. Cell Dev. Biol., 2016, 4, 1-12.
53 J. E. Nagel, G. D. Collins and W. H. Adler, Cancer Res., 1981, 41, 2284-2288.
54 X. Song, C. Xu, X. Wu, X. Zhao, J. Fan and S. Meng, Biologicals, 2020, 68, 46-53.
55 S. Neri, E. Mariani, A. Meneghetti, L. Cattini and A. Facchini, Clin. Diagn. Lab. Immunol., 2001, 8, 1131-1135.

1. A microfluidic device for single cell analysis and isolation, the device comprising:
(a) an aqueous microdroplet generator comprising an oil inlet, one or more cell suspension inlets, and a flow focusing junction capable of forming a stream of aqueous microdroplets in the oil under continuous flow, the aqueous microdroplets containing the one or more cell suspensions;
(b) a flow layer comprising:
(i) a flow channel, wherein the flow channel accepts the stream of aqueous microdroplets from the aqueous microdroplet generator at a first end and terminates in an outlet port at a second end;
(ii) a plurality of docking stations for the aqueous microdroplets, the docking stations arranged in a plurality of parallel rows, each docking station comprising an entry port and an exit port, each docking station fluidically coupled to the flow channel through its entry port; and the docking stations configured for light microscopic observation; and
(iii) a plurality of cell extraction channels, each cell extraction channel aligned in parallel to one of said rows of docking stations, each cell extraction channel coupled to each docking station of its aligned row of docking stations through the respective exit ports of the coupled docking stations, wherein each cell extraction channel terminates in first and second extraction outlets disposed at opposite ends of the cell extraction channel;
(c) a control layer comprising a plurality of parallel aligned control channels, each control channel comprising a plurality of microvalves and a pressure control port, wherein the control channels are aligned at right angles to said rows of docking stations, and wherein each microvalve is superimposed over one of said docking stations;
(d) a deformable membrane disposed between the flow layer and the control layer; wherein pressure of a fluid in a selected one of the control channels controls an activation state of the microvalves in said one of the control channels, and wherein release of an aqueous microdroplet from a docking site into the corresponding extraction channel is determined by the activation state of the microvalve overlapping the docking site and by a flow rate of oil in the flow channel.

2. The microfluidic device of claim 1, wherein the aqueous microdroplet generator comprises two or more cell suspension inlets leading to a merging junction for mixing of two or more cell suspensions.

3. The microfluidic device of claim 1, wherein the aqueous microdroplet generator is embedded within the flow layer.

4. The microfluidic device of claim 1, wherein the flow channel has a serpentine configuration having two or more linear sections arranged in parallel to one another, each linear section aligned with a row of docking stations, and wherein the linear sections are connected by curved sections of the flow channel not associated with docking stations.

5. The microfluidic device of claim 1, wherein the rows of docking stations contain from about 10 to about 100 docking stations per row.

6. The microfluidic device of claim 1, wherein the device contains from about 4 to about 20 rows of docking stations.

7. The microfluidic device of claim 1, wherein the device contains from about 40 to about 2000 docking stations.

8. The microfluidic device of claim 1, wherein the control channels and microvalves are configured for operation by a pressure-controlled gas introduced at the pressure control port.

9. The microfluidic device of claim 1, wherein each aqueous microdroplet in a docking station can be individually extracted by a combination of microvalve actuation and extraction channel flow.

10. The microfluidic device of claim 1, wherein the flow layer is housed in a first PDMS slab which is bonded to a glass substrate on one side and bonded on another side to a first side of the membrane, and wherein the control layer is housed in a second PDMS slab bonded to a second side of the membrane opposite to the first side.

11. The microfluidic device of claim 1, wherein the membrane comprises PDMS.

12. The microfluidic device of claim 1, wherein the membrane has a thickness of about 30-50 microns, such as about 40 microns

13. The microfluidic device of claim 1, wherein the aqueous microdroplet generator generates aqueous microdroplets having a diameter from about 150 microns to about 200 microns.

14. The microfluidic device of claim 1, wherein each microdroplet docking site has a diameter of about 200 microns.

15. The microfluidic device of claim 1, wherein the entry ports and exit ports of the aqueous microdroplet docking stations are configured to allow entry of aqueous microdroplets into all docking stations and their retention in the docking stations under a baseline oil flow condition, and to allow exit of an aqueous microdroplet only under higher oil flow induced by actuation of the microvalve overlapping the docking station housing the microdroplet.

16. The microfluidic device of claim 1, wherein the device is capable of isolating, analyzing, and delivering live individual cells of interest from a population of cells.

17. A system for single cell analysis and isolation, the system comprising:
(a) the microfluidic device of any of the preceding claims;
(b) fluid delivery devices to provide flow of oil into the oil entry port, one or more cell suspensions into the one or more cell suspension entry ports, and oil into the extraction channels;
(c) a microscope for observing cells in the aqueous microdroplets in the docking stations; and
(d) an imaging system for recording and analyzing images of cells obtained with the microscope.

18. The system of claim 17, wherein the microscope is an inverted fluorescence microscope.

19. The system of claim 17, wherein the system is capable of unattended, programmed analysis and extraction of cells of interest.

20. The system of claim 17, further comprising a cell culture system for culturing and/or expansion of cells isolated from aqueous microdroplets extracted from the microfluidic device.

21. A method of single cell analysis and isolation, the method comprising the steps of:
(a) providing the microfluidic device of claim 1;
(b) loading a plurality of individual cells into aqueous microdroplets in an oil stream using the microfluidic device;
(c) directing the microdroplets into the docking sites of the microfluidic device;
(d) incubating the docked microdroplets for a period of time and observing cell behaviour in the docked microdroplets, whereby individual microdroplets containing one or more cells of interest are identified; and
(e) extracting microdroplets containing one or more cells of interest by actuating one or more microvalves of the microfluidic device associated with docking stations containing the one or more cells of interest, and directing the extracted microdroplets to a collection device through one or more extraction outlets of the device.

22. The method of claim 21, wherein step (e) comprises using an oil flow rate in the flow channel that is slow enough to allow cells to remain trapped within docking stations unless and until a microvalve is actuated.

23. The method of claim 22, wherein actuation of a microvalve associated with a selected docking station causes an increase in oil flow rate through the docking station, whereby the aqueous microdroplet in the docking station moves out of the docking station through the docking station exit port and into an extraction channel.

24. The method of claim 21, wherein two different cell populations are mixed, and aqueous microdroplets are formed containing a mixture of the two different cell populations.

25. The method of claim 24, wherein an interaction between cells from the two different cell populations are analyzed and the results used as a basis for selecting aqueous microdroplets for extraction.

26. The method of claim 25, wherein the two different cell populations comprise immune effector cells and target cells of the immune effector cells.

27. The method of claim 26, wherein the effector cells are natural killer (NK) cells and the target cells are cancer cells or cells infected with a microbial pathogen, such as a virus.

28. The method of claim 26, wherein the effector cells and target cells are obtained from the same subject.

29. A method of immunotherapy of a subject, the method comprising the steps of:
(a) providing the device of claim 1 and samples of effector cells and target cells from the subject.
(b) loading the effector cells and target cells into docking stations of the device, whereby aqueous microdroplets are formed comprising both one or more effector cells and one or more target cells;
(c) observing behaviour of the cells for a period of time, whereby microdroplets are identified containing cells with desired actions by an effector cell against a target cell;
(d) extracting the identified microdroplets;
(e) isolating effector cells of interest from the extracted microdroplets;
(f) culturing the effector cells to expand their number; and
(f) administering the expanded effector cells to the subject.

30. The method of claim 29, wherein the subject has cancer, the effector cells are NK cells of the subject, and the target cells are cancer cells of the subject.
